(19) United States
(12) Patent Application Publication
(10) Pub. No.: US 2024/0074700 A1
(43) Pub. Date: Mar. 7, 2024

(54) HEADGEAR FOR BIOMETRIC STUDY

(71) Applicants: Mohammad Mehdi Davari, Isfahan (IR); Masih Davari, Isfahan (IR)

(72) Inventors: Mohammad Mehdi Davari, Isfahan (IR); Masih Davari, Isfahan (IR)

(21) Appl. No.: 18/261,902

(22) PCT Filed: Jan. 29, 2021
(86) PCT No.: PCT/IB2021/050735

§ 371 Date: Jul. 18, 2023

(51) Int. Cl. A61B 5/00; A61B 3/14; A61B 5/16; A61B 5/291

(52) U.S. Cl. CPC A61B 5/6803; A61B 3/14; A61B 5/163; A61B 5/291

(57) ABSTRACT

Disclosed is a headgear for biometric research on human subjects. The headgear includes a frame that can be worn around the head. One or more of bridges extends from the frame over the scalp. Each bridge has one or more electrode housings coupled to the bridge. Each housing having an electrode for measuring the brain's electrical activity. Each bridge can pivotally couple to the frame for limited movement of the bridge relative to the frame. The headgear further comprises one or more forehead bridges that extends from the frame towards the middle of the eye. Tip of the forehead bridge having a sensor unit for tracking the movements of the eyes.

### FIELD OF INVENTION

The present invention relates to a headset and more particularly, the present invention relates to a headset with integrated electroencephalography and eye-tracking sensors.

### BACKGROUND

A biometric study is a research that aims to explore and understand the human's cognitive and emotional behavior using different biometric devices to capture signals from a person. Biometric devices are those that are helping to capture human vital and physiological signals. These signals include brain signals captured from the brain, visual signals captured from eye movements and pupils, respiratory signal captured from breathing, heart rate captured from the chest, body temperature, etc.

A typical biometric study may include several types of equipment, a few of which can be bulky. The different types of equipment are used to measure different biometrics or mental state. The number of equipment must be used at the same time to gain insights about the interrelation of different factors affecting the mental state. EEG devices are commonly used to study brain electrical activity. EEG headsets generally include a number of electrodes that are placed in different positions over the scalp. Standard practiced can be followed for locating the precise position for attaching the electrodes. Similarly, other electrodes or sensors can be used to measure different psychological and physiological parameters depending upon the objective of the study.

Although more data is always better for research, however, several types of equipment must be used which may be discouraging for the participants of the study or patients. Thus, a need is appreciated for compact devices that can simultaneously measure two or more parameters and are easy to wear.

### SUMMARY OF INVENTION

The principal object of the present invention is therefore directed to a wearable headgear with multiple electrodes or sensors for measuring two or more parameters.

It is another object of the present invention that the disclosed headgear can easily fit different head sizes and shapes.

It is still another object of the present invention that the disclosed headgear can adjust to the eye positions.

It is a further object of the present invention that the disclosed headgear has enough degree of freedom of movement.

In one aspect, disclosed is headgear for biometric research on human subjects. The disclosed headgear is a portable headgear that can be used in both laboratory, home, and office settings. The headgear is flexible enough to allow fitting different head sizes. The headgear includes a frame that can be worn around the head. A plurality of bridges extends from the frame over the scalp. Each bridge has one or more electrode housings slidably coupled to the bridge. Each housing having an electrode for detecting the brain's electrical activity. Each bridge can pivotally be coupled to the frame for limited movement of the bridge relative to the frame. The headgear further comprises a forehead bridge that extends from the frame. The forehead bridge extends from the frame to the middle of the eye and the tip of the forehead bridge further having one or more optical sensors for tracking the movement of the two eyes.

In one aspect, the length of the bridges can be adjusted. Each bridge can include one or more sections connected through an expandable joint that allows the sections to extend and retract respective to each other. The bridges can be removed and replaced. Similarly, electrode housings can be removed and replaced. The forehead bridge can be adjusted in length. Moreover, the forehead bridge can be removed and replaced.

These and other objects and advantages of the embodiments herein will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of the headgear, according to an exemplary embodiment of the present invention.

FIG. 2 shows another embodiment of the headgear, according to an exemplary embodiment of the present invention.

FIG. 3 shows a bridge having the electrode housing, according to an exemplary embodiment of the present invention.

FIG. 4 shows another embodiment of the headgear, according to an exemplary embodiment of the present invention.

### DETAILED DESCRIPTION OF THE INVENTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

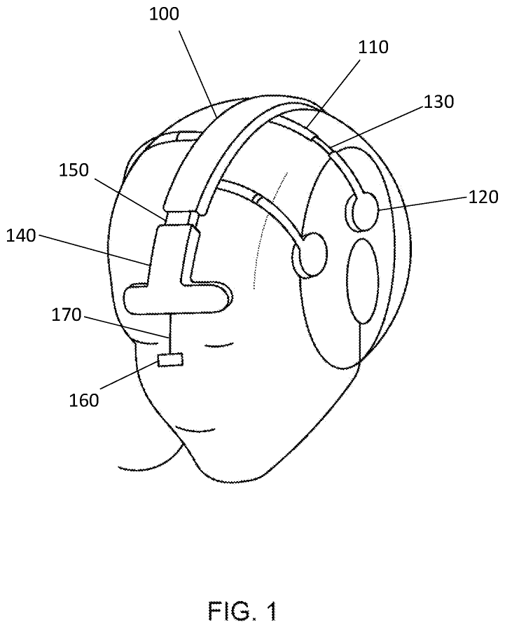

Referring to FIG. 1, which shows an exemplary embodiment of the headgear worn over the head. The headgear includes a frame 100 that goes over the top of the head. Three bridges 110 can be seen extending out from the frame 100. The frame can include multiple slots along its length. One or more of such bridges can be coupled to the frame at these slots. The bridges run over the scalp of the head and support the EEG electrodes. Since the position of the EEG electrodes must be adjusted, the frame allows coupling the bridges at different positions i.e., slots. The slots of the frame can also provide a limited lateral movement to the bridges over the scalp. Each bridge can include two or more sections connected through an expandable joint 130. Each section can be extended and retracted respectively to the adjacent sections. Therefore, disclosed is a versatile headgear that allows removing, replacing, and adjusting the bridges for accurate positioning of the electrodes.

FIG. 1 further shows electrode housing 120 at the end of the bridges. The electrode housing 120 includes EEG electrodes for measuring the electrical activity of the brain. The electrode housing can be slidably mounted to the bridges that allow adjusting the positions of the electrodes on the scalp. A forehead bridge 140 can also be seen in FIG. 1 extending from the front end of the frame. The forehead bridge extends towards the middle of the eyes. The forehead bridge can be connected to the frame through an expandable joint 150 that may allow adjusting the length of the forehead bridge. At the end of the bridge can be seen a sensor unit 160 that extends from the forehead bridge through a shaft. The sensor unit includes one or more eye-tracking sensors, such as optical sensors. In one implementation, the sensor unit can include two sensors for each eye. The shaft connecting the sensor unit to the forehead bridge can be semirigid i.e., rigid enough to hold the sensor unit in position and flexible to allow adjusting the position of the sensor unit relative to the eyes. Moreover, the forehead bridge can be slidably coupled to the frame that may allow lateral movement of the forehead bridge. The forehead bridge can slightly move in left and right directions for perfect fit. For the slidably coupled, a joint attaching the forehead bridge to the frame can be a longitudinal slot and the forehead bridge can slide along the slot. Alternatively, multiple attachment points for the forehead bridge can be provided on the frame. The attachment points can be closely spaced for minor adjustments of the forehead bridge. The forehead head bridge can be removed from one attachment point and attached to another attachment point. Additionally, more than one forehead bridge is within the scope of the present invention.

Disclosed headgear can be easily adjusted to different head sizes and head shapes. Moreover, the forehead bridge with the optical sensor can be easily adjusted to different eye positions on the face. In a preferred embodiment, the disclosed headgear is unified, modular, adjustable, and flexible hardware that integrates the functionality of EEG and eye-tracking (ET). Disclosed headgear is advantageous by providing the freedom and flexibility of setting sensors in different positions and the possibility of having different combinations of sensors in one frame.

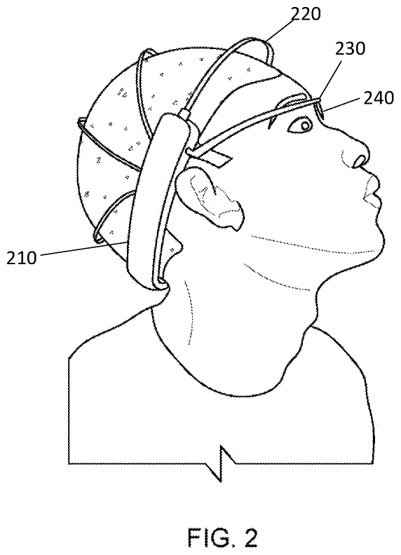

FIG. 2 shows another embodiment of the headgear worn over the head. The headgear includes a frame 210 that is worn around the back of the head and adjacent to the ears. Four bridges 220 can be seen extending from the frame over the scalp of the wearer's head. A forehead bridge 230 also extends from the end portions of the frame towards the middle portion of the eye. A sensor unit 240 positioned on the forehead bridge near the middle of the eyes can track the movements of the eyes.

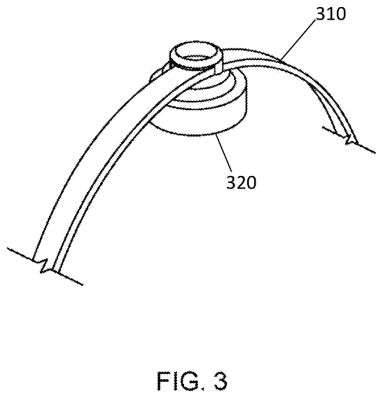

FIG. 3 shows an exemplary embodiment of the electrode housing mounted on bridge 310. The electrode housing 320 includes a base portion that has an EEG electrode. The top of the electrode housing has a slot and a cap, wherein the bridge passes through the slot. The electrode housing can slide over the bridge. The cap can be provided with a mechanism to lock the electrode housing over the bridge.

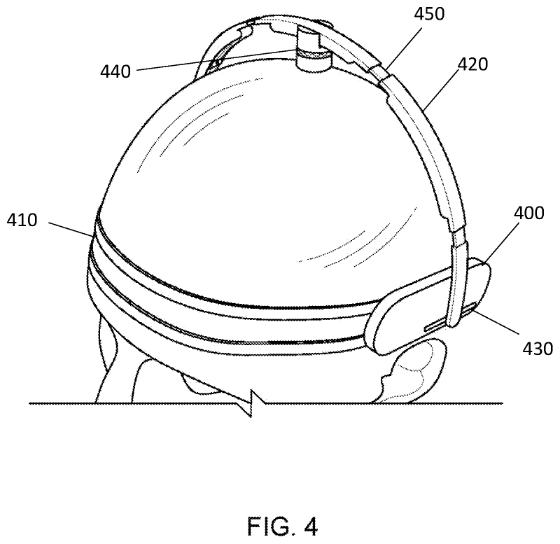

FIG. 4 shows another embodiment of the headgear having a pair of frames 400 positioned above and adjacent to the two ears. A pair of elastic bands 410 can be seen connecting the two frames. The two elastic bands 410 can be used to wear the headgear over the head. The elastic bands keep the two frames over the head, wherein the elasticity of the elastic bands allows the headgear to fit the heads of different shapes and sizes. Each frame 400 includes a longitudinal slot 430. A bride 420 can be seen slidably mounted to the longitudinal slot 430. The two ends of the bridge coupled to the longitudinal slot of the two frames and the bridge runs over the head. The position of the bridge can be adjusted over the head by sliding through the longitudinal slot. Moreover, the bridge can be removed and replaced by another bridge. An electrode housing 440 can be seen slidably coupled to the bridge. The desired number of electrode housing can be coupled to the bridge. The disclosed headgear can be customized by adding and replacing the components, such as the bridges, electrode housing, the forehead bridge, and the sensor unit making the headgear versatile. The bridge shown in FIG. 4 has expandable joints 450 that allow adjusting the length of the bridge. Thus, depending on the requirement, one or more bridges can be added to a frame. Similarly, one or more electrodes can be coupled to the bridge. The position of the electrodes over the scalp can be adjusted by tweaking the position of the bridge and the electrode housing. The elasticity and expandability of the bridges allow the headgear to easily adjust for wearing over the head. The forehead bridge can also be removed and reinstalled as and when required. The sensor units can be replaced with a different sensor.

It is to be understood that the above disclosure has been described using electrodes to measure brain activity. Any other sensor to measure brain activity can be incorporated without departing from the scope of the present invention. For example, Functional near-infrared spectroscopy (fNIRS) is a known neuroimaging technique. Moreover, the drawings show the sensor unit or camera adjacent to the middle of the eyes. However, the camera can be positioned anywhere without departing from the scope of the present invention provided that the cameras can track the movement of eyes. Additionally, more than one sensor unit or camera can be used to track the movement of both eyes. For example, two cameras can be used i.e., a left camera and a right camera for tracking left eye and right eye movements, respectively. Generally, an eye line is describing herein as an imaginary line that extends from the center of the left eye up to the right eye. The forehead bridge can extend near this eye line. Perhaps two or more forehead bridges can extend towards the eye line. Perhaps a left forehead bridge extends nearby the left eye and a right forehead bridge extends towards the right eye, i.e., a slight height above the eyes. The left camera and the right camera coupled to the tips of the left forehead bridge and the right forehead bridge can face the left eye and the right eye, respectively.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A headgear assembly comprising:
frame configured to be worn over a wearer's head;
at least one bridge pivotally and removably coupled to the frame, the at least one bridge runs over the scalp of the head;
at least one housing slidably mounted to the at least one bridge, the at least one housing having one or more sensors for measuring the brain activity;
one or more forehead bridges coupled to the frame and extends towards an eye line, the eyeline extending between a left eye and a right eye; and
one or more cameras coupled to the one or more forehead bridges and configured to track the movement of the left eye and the right eye.

2. The headgear according to claim 1, wherein the bridge is comprised of a plurality of sections coupled through expandable joints, each section of the plurality of sections can be extended and retracted relative to the adjacent section.

3. The headgear according to claim 1, wherein the one or more forehead bridges are coupled to the frame through expandable joints permitting the one or more forehead bridges to be adjusted in length.

4. The headgear according to claim 1, wherein the one or more forehead bridges are slidably coupled the frame permitting lateral movement of the one or more forehead bridges relative to the frame.

5. The headgear according to claim 1, wherein the frame includes a plurality of closely spaced attachments points, arranged in a horizontal axis, for attaching the one or more forehead bridges.

6. The headgear according to claim 1, wherein the headgear comprises two to six bridges pivotally coupled to the frame.

7. The headgear according to claim 1, wherein the one or more cameras are coupled to the one or more forehead bridge through a semi-rigid shaft that allows adjusting the position of the one or more cameras relative to the eyes.

8. The headgear according to claim 1, wherein the housing is having a base that includes an electrode, a slot is configured over the base and allows a bridge to pass through, wherein the housing slides over the bridge.

9. The headgear according to claim 8, wherein the housing further having a cap configured to lock the movement of the housing over the bridge.

10. The headgear according to claim 1, wherein the frame is having a plurality of slots along its length, the at least bridge is interchangeably coupled to any of the plurality of slots.

11. The headgear according to claim 1, wherein a left forehead bridge and a right forehead bridge extends towards the left eye and the right eye respectively, a left camera is coupled to the left forehead bridge and a right camera is coupled to the right forehead bridge.

12. The headgear according to claim 1, wherein the one or more sensors of the housing are EEG electrodes.
